(19) United States
(12) Patent Application Publication
(10) Pub. No.: US 2020/0405524 A1
(43) Pub. Date: Dec. 31, 2020

(54) CARRIER FOR URINE BAG

(71) Applicant: AKAAL INNOVATIONS SDN BHD, Selangor (MY)

(72) Inventor: Gurvinder Singh GILL, Selangor (MY)

(21) Appl. No.: 16/478,862

(22) PCT Filed: Mar. 20, 2019
(86) PCT No.: PCT/MY2019/050023

§ 371 Date: Jul. 18, 2019

(30) Foreign Application Priority Data
Mar. 20, 2018 (MY) PI 2018701085

(51) Int. Cl. A61F 5/44

(52) U.S. Cl. CPC A61F 5/4408

(57) ABSTRACT

The present invention relates to a carrier for urine bag, wherein the carrier holds the urine bag firmly over the body of the patient. The carrier also holds the catheter tube stably with the help of a plurality of tube holders present in the carrier body, thus allowing an easy movement of the patient, keeping the patient's hands free and allowing the patient to do his daily activities without any pain and guilt. The carrier comprises a carrier body 100, at least two extended arms 130, a pocket 101, an opening 201, a plurality of tube holders 108, a belt 120 and a connecting means 114.

### FIELD OF INVENTION

The present invention generally relates to urine bag supporters and particularly to carriers for urine bag. The present invention more particularly relates to a urine bag carrier that firmly and stably holds the urine bag over a body of a patient without disturbing the tube of the catheter and preventing pain in patient.

### BACKGROUND OF THE INVENTION

Individuals diagnosed and afflicted with certain urological conditions that cause blockage of the urinary tract or are otherwise afflicted with the inability to discharge urine voluntarily are usually provided with a device that collects and stores urine. This device is a bag made of flexible elastic material such as vinyl. This bag has an inlet orifice that is connected by an elastomeric tube, to a catheter and an outlet orifice that is attached to a drainage valve. The catheter is a thin flexible tube that is inserted into the body, usually through the urethra and is threaded into the bladder. This allows the bladder to drain urine via the catheter into the bag. The bag is more commonly known as a urine bag. The drainage valve is configurable to drain urine collected in the bag from time to time and is usually made of a rigid plastic material. Collectively, the combination of the bag, elastomeric tube connected to the catheter via the inlet orifice of the bag and the drainage valve connected to the outlet orifice of the bag has come to be known simply as a urine collection bag or urine bag and is an indispensable apparatus that is used in medical care.

The conventional urine bag imposes a mental load or stress on the patient as the patient has to be careful not to exert unduly excessive amounts of pull and tug on the catheter attached to the urine bag due to movement of the urine bag that results from movement of the patient, in order to prevent the occurrence of trauma to the urinary tract and hence prevent experiencing pain. Hospital patients or individuals fitted with a urine bag by way of a catheter, need to be extra careful when ambulating, as the patient has to hand-carry the urine bag and prevent the elastomeric tube connecting the bag to the catheter from being inadvertently being entangled with an article and hence preventing the catheter from tearing away from the urinary tract and causing trauma to the urinary tract. Hence, the use of a urine bag, one can surmise, substantially hinders a patient's mobility, particularly when ambulating.

Considering the above facts, the prior arts have urine bags developed with a bag holding device attached with it. This bag holding device comprises of a main frame body having a proximal end disposed with a pliable clip configured to attach to an article of clothing or a bed frame of a hospital bed; and a distal end having end members that are configured to attach to the urine bag. However, such an arrangement, does not provide optimum comfort to the patient. Any movement of the patient even when resting on a hospital bed may result in pulling and tugging of the catheter attached thereto. Such constant tugging and pulling of the catheter may result in irritation and possibly even trauma to the bladder, which in turn may cause chronic bleeding,

Particularly one prior art solution, entails securing a urine bag fitted to a patient on the patient's thigh by means of a pouch including two circumferential elastic straps displaced from one another by a predetermined spatial distance. The disadvantage is that these elastic straps tend to be displaced from an original position as the urine bag fitted in the pouch fills up and becomes heavier and as a consequence results in pulling and tugging of the catheter inserted into the urinary tract of the person fitted with the urine bag which hence may in turn result undesirably in the inflicting of trauma to said person's urinary tract.

Further, during the use of these bags, there is movement of the catheter tube while the patient makes a small move. This leads to severe pain and trauma to the urinary bladder of the patient.

Hence, in view of the foregoing, there is a need to develop a carrier for a urine bag having arrangement for firmly and stably holding the urine bag and further having arrangement for stably holding the tube of the catheter, because of which the patient can move and walk freely without disturbing the catheter and the tubes.

### SUMMARY OF THE INVENTION

Thus, the primary object of the present invention is to provide a carrier for urine bag for firmly and stably holding the urine bag and the tubes intact.

Another object of the present invention is to provide a carrier for urine bag that helps the patient walk freely without holding the urine bag with hands and which keeps the catheter tubes intact without causing any pain to the patient during movements.

Yet another object of the present invention is to provide a carrier for urine bag that is capable of securely accommodating a urine bag even when the urine bag is close to being filled to full fluid retaining capacity or is filled to full fluid retaining capacity by accommodating the outwardly projecting expansion and laterally projecting expansion of the filled urine bag.

Yet another object of the present invention is to provide a carrier for urine bag that allows for a substantially clear view of the volume of urine retained within a urine bag housed therein, to a caregiver or patient that is fitted with the urine bag and said urine bag carrier.

Yet another object of the present invention is to provide a carrier for urine bag that is capable of housing a wide variety of urine bags that are different from one another by minor points of detail such as fluid retaining capacity (i.e. size) and other auxiliary features.

Yet another object of the present invention is to provide a carrier for urine bag that is not constrained to use in one leg and can further be easily repositioned to prevent or mitigate discomfort experienced by the patient due to long and extended use that results from humidity and moisture build-up between the skin and the material of the carrier.

Yet another object of the present invention is to provide a carrier for urine bag that is highly effective, comfortable to wear, easy to adjust, and inexpensive in acquisition and upkeep.

According to the embodiments of the present invention, a carrier 200 for urine bag is provided. The carrier for urine bag comprises a carrier body 100, at least two extended arms 130, a pocket 101, an opening 201, a plurality of tube holders 108, a belt 120 and a connecting means 114. The at least two extended arms 130 are present on the carrier body 100. The pocket 101 is present in the carrier body 100 for placing a urine bag 300. The pocket 101 is formed by a pair of horizontally present parallel supporting means 103 and a plurality of vertical supporters 106 on a front side, and a rear panel 104 on a rear side. The opening 201 is present at the bottom of the carrier body 100, according to an embodiment of the present invention. The opening 201 is present anywhere in the carrier body, as per the requirement for the drainage valve adjustment, and for passaging of a drainage valve. The plurality of tube holders 108 are present over the corners of the carrier body 100. The connecting means connects the carrier body 100 with the belt 120.

According to an embodiment of the present invention, the at least two extended arms 130 are a top extended arm and a bottom extended arm. The at least two extended arms 130 are wrapped around a patient's body and are fixed with the help of a fastening means present on the horizontal supporting means 103 of the carrier body 100.

According to an embodiment of the present invention, the pocket 101 is covered with a netting fabric internally.

According to an embodiment of the present invention, the connecting means 114 comprises a distance adjusting mechanism 116 for adjusting the distance between the belt 120 and the carrier body 100.

According to an embodiment of the present invention, the connecting means 114 further comprises a tube holder 115 in the centre for holding and passaging of the tube of the urine bag 300.

According to an embodiment of the present invention, the pair of horizontally present parallel supporting means 103 are made up of a flexible material to help in holding the urine bag firmly.

According to an embodiment of the present invention, the urine bag 300 is fastened firmly inside the pocket 101 with the help of fastening means.

According to an embodiment of the present invention, the fastening means comprises a velcro arrangement, snap-push buttons, or hook fasteners.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

Other objects, features, and advantages of the invention will be apparent from the following description when read with reference to the accompanying drawings. In the drawings, wherein like reference numerals denote corresponding parts throughout the several views:

FIG. 1 shows a front view of the carrier for urine bag, according to an embodiment of the present invention.

FIG. 2a shows a front view of the carrier for a urine bag and FIG. 2b is shows a rear view of the carrier for a urine bag, according to an embodiment of the present invention.

FIG. 3a is a front view of the carrier for a urine bag and FIG. 3b is a rear view of the carrier for a urine bag, according to an embodiment of the present invention.

FIG. 4 shows a carrier for a urine bag worn on a thigh of a patient and containing as well as securing a urine bag to said thigh of the patient, according to an embodiment of the present invention.

FIG. 5 shows carrier for a urine bag worn on a thigh of a patient showing the tube holders and different settings of holding the tube intact in the carrier, according to an embodiment of the present invention.

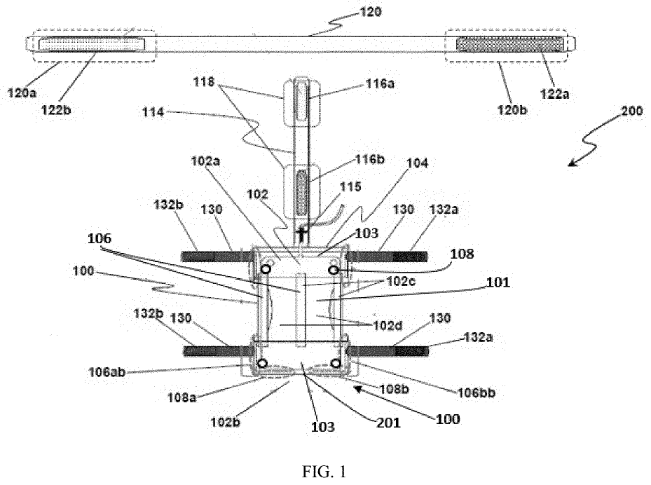

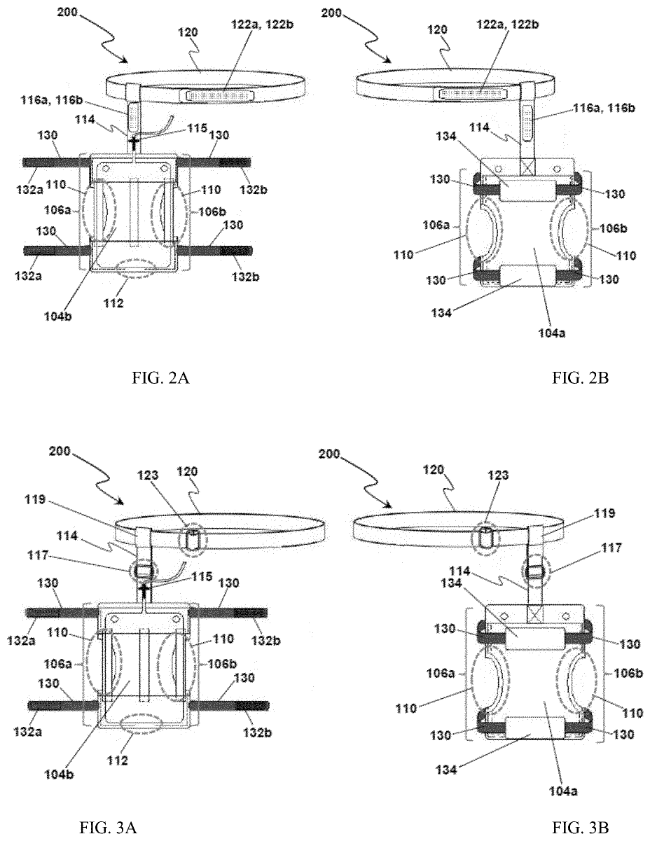

### DETAILED DESCRIPTION OF THE PREFERRED EMBODIMENTS

The present invention will now be described in detail with reference to the accompanying drawings.

The various embodiments of the present invention provide a carrier for urine bag 200. The carrier 200 for urine bag comprises a carrier body 100, at least two extended arms 130, a pocket 101, a 201, a plurality of tube holders 108, a belt 120 and a connecting means 114. The at least two extended arms 130 are present on the carrier body 100. The pocket 101 is present in the carrier body 100 for placing a urine bag 300. The pocket 101 is formed by a pair of horizontally present parallel supporting means 103 and a plurality of vertical supporters 106 on a front side, and a rear panel 104 on a rear side. The opening 201 is present at the bottom of the carrier body 100 for passaging of a drainage valve, according to an embodiment of the present invention. The opening 201 is present anywhere in the carrier body, as per another embodiment herein, for an easy passage of the drainage valve, and setting of the catheter tube. The plurality of tube holders 108 are present over the corners of the carrier body 100. The connecting means connects the carrier body 100 with the belt 120.

According to an embodiment of the present invention, the carrier 200 for a urine bag 300 is configured to secure a urine bag 300 to a thigh of a person which can be worn as an undergarment or otherwise. A pocket 100 is capable of securely housing and accommodating a urine bag 300 even when the urine bag 300 is close to being filled to full fluid retaining capacity or is filled to full fluid retaining capacity.

According to an embodiment of the present invention, the at least two extended arms 130 are a top extended arm and a bottom extended arm. The at least two extended arms 130 are wrapped around a patient's body and are fixed with the help of a fastening means present on the horizontal supporting means 103 of the carrier body 100.

According to an embodiment of the present invention, a plurality of slots 102d allow a clear view of a level of urine contained in a urine bag 300 housed in the pocket 101 of the carrier 200, to a patient fitted with said carrier 200 or a caregiver of said patient.

According to an embodiment of the present invention, the front panel 102 further comprises a netting fabric that spans the longitudinal distance between the pair of strips of flexible material 102a, 120b. The netting fabric is substantially transparent and allows a substantially clear view of the volume of urine contained in a urine bag 300 housed within the pocket of the carrier 100, to a patient fitted with the urine bag 300 or a care-giver.

According to an embodiment of the present invention, the pocket 101 is covered with a netting fabric internally. The netting fabric is configured to accommodate stretching of the plurality of longitudinally extending flexible elastic strips 102c and hence acts in cooperation with said longitudinally extending flexible elastic strips 102c to accommodate outward projecting expansion of a urine bag 300 housed/contained within the pocket 101 of the carrier 200.

According to an embodiment of the present invention, the carrier 200 for a urine bag 300 is advantageous because the carrier bag securely contains and secures a urine bag 300 irrespective of a percentage of its full fluid volume retaining capacity that is utilized and hence mitigate if not prevent the incidence of the urine bag from falling off, and hence inadvertent urinary tract trauma to a patient or person fitted with a urine bag 300 and consequently discomfort experienced by the patient or person as a result of being fitted with the urine bag 300. The carrier bag allows a substantially clear view of the volume of fluid retained within a urine bag contained or housed within the pocket 101, to the patient or person fitted with the urine bag 300 himself or herself, or alternatively a care-giver.

The surprising advantages are achieved by the novel configuration of the front panel 102 of the pocket 101 of the carrier 200 for a urine bag 300. More particularly, the arrangement of the front panel 102 of said pocket 101 as described in a preceding paragraph above, caters not only to the outwardly projecting expansion of a urine bag 300 contained within the pocket 101, but further accommodates the laterally projecting expansion of the urine bag 300 as it is being filled or is filled at its full fluid retaining capacity, via the lateral openings 110. The side lateral openings 110, according to an embodiment of the present invention, are not stitched, in order to accommodate more expansion as the urine bag is filled with more fluid. According to another embodiment of the present invention, the side lateral openings 110 are stitched with a different fabric, wherein the fabric is stitched on top and bottom but crumpled in the middle portion, in order to allow more expansion of the urine bag.

Also, the side openings are curved to allow the bag to conform to the leg, this helps reduce any bulges or excess material pointing out, showing through the patient's pants. The configuration of the front panel 102 described in a preceding paragraph above, further allows for a substantially clear view of the fluid content of a urine bag 300 housed within the pocket 101 of the carrier 200.

According to an embodiment of the present invention, the connecting means 114 comprises a distance adjusting mechanism 116 for adjusting the distance between the belt 120 and the carrier body 100. The belt 120 of flexible material for encircling a body part of a patient or person and supporting a weight of a fully filled urine bag 300 or otherwise contained within the pocket 101 via a strap 114 of longitudinally extending flexible material extending from the rear panel 104 of the pocket 101.

According to an embodiment of the present invention, the belt 120 is a waist encircling belt 120 (as shown in figures). The waist encircling belt 120 has its free ends portions 120a, 120b disposed with complementarily acting portions of an adjustable fastening mechanism, that act in cooperation to allow adjustable and removable circumferential securement of said waist encircling belt 120 onto a patient's or person's waist based on said patient's or person's waist measurement and comfort level. The flexible material of the waist encircling belt 120 and the longitudinally extending strap 114 is an orthopaedic woven cotton canvas fabric, which is known to be hypo-allergenic and thus mitigates if not prevents the occurrence of skin irritation and rashes on a portion of skin of a patient or person fitted with the carrier 200. The strap of flexible material 114 that extends longitudinally from the rear panel 104, extends longitudinally from a strip of flexible material forming a top portion 104c of the rear panel 104 and is attached to said top portion 104c by way of seams. The longitudinally extending strap of flexible material 114 extending from the rear panel 104 of the pocket 101 serving to enable securing of the pocket 101 of the carrier 200 for a urine bag 300 in accordance to an embodiment of the present invention, to the waist encircling belt 120 and hence effectively transmits the downward force exerted by the weight of a filled urine bag 300 contained within the pocket 101 of the carrier 200 to the waist belt 120 which is worn around the waist of a patient or person and hence the waist of the patient or person, effectively supports the bulk of the weight of said urine bag 300 (filled to full fluid retaining capacity or otherwise).

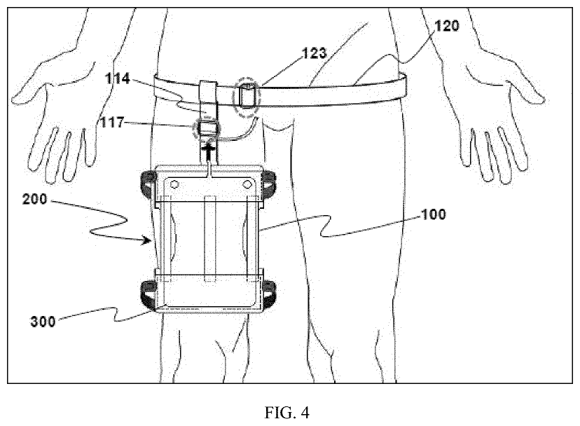

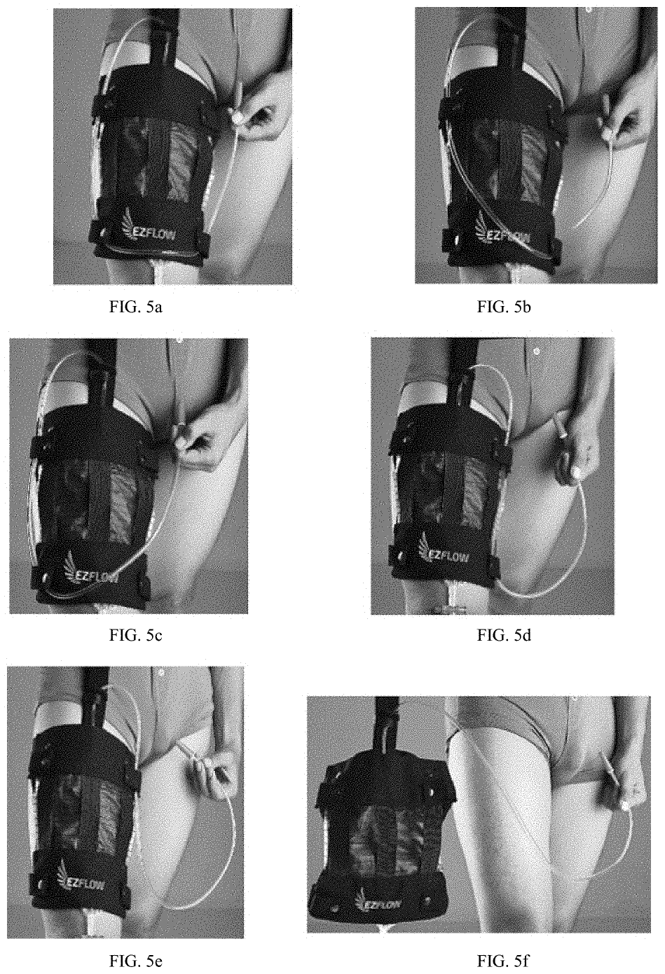

According to an embodiment of the present invention, the strap of flexible material 114 extending longitudinally from the rear panel 104 of the pocket 101 has an adjustable fastening means or arrangement that is configured to enable adjustably securing the pocket 101 via said longitudinally extending strap of flexible material 114 to the waist belt 120 and hence waist of a patient or person fitted with a urine bag 300. Specifically, in accordance to an embodiment, the adjustable fastening means or arrangement comprises a slip buckle 117 (as shown in FIG. 3 and FIG. 4) that acts in cooperation with a moveable cotton orthopaedic belt loop 119 that encircles a portion of the waist belt 120 longitudinally.

According to an embodiment of the present invention, the longitudinally extending strap of flexible material 114, extending longitudinally from the rear panel 104 of the pocket 101, has one end, attached to the rear panel 104 and specifically in accordance to an exemplary embodiment of the carrier 200 of the present invention, is attached to a strip of flexible material forming a top portion 104c of the rear panel 104 of the pocket 101 by way of seams. The remaining portion, i.e. a portion of the strap 114 that follows a free end of the strap 114, is disposed with an adjustable fastening means or arrangement.

According to an embodiment of the present invention, the adjustable fastening means or arrangement is a slip buckle 117. Specifically, the free end of the longitudinally extending strap of flexible material 114 is fed through a loop portion (not explicitly shown in the appended set of figures) of the slip buckle 117, while a portion of the moveable orthopaedic belt loop 119, longitudinally encircling a portion of the waist belt 120, is also fitted to a hook portion (not explicitly shown in the appended set of figures) of the slip buckle 117.

According to an embodiment of the present invention, the adjustable fastening means or arrangement comprises a plurality of hook fasteners 116a and a plurality of loop fasteners 116b that are disposed at symmetrically opposing portions 118 of the strap 114. The plurality of hook fasteners and plurality of loop 30 fasteners 116a, 116b acts in cooperation to enable the adjustable securing of the pocket 101 containing a urine bag 300 to the waist belt 120 secured in snug surface communication with a patient's waist. The longitudinally extending strap of flexible material 114 is a strap of 1.5″ orthopaedic cotton strapping. More particularly, the longitudinally extending strap of flexible material 114 comprises woven orthopaedic cotton canvas fabric. According to an embodiment of the present invention, the longitudinally extending strap of flexible material 114 is disposed at an end distal to a free end, with a tube retaining member 115. The tube retaining member 115 comprises a strip of flexible material composed of woven orthopaedic cotton canvas fabric (exemplarily) that is remove ably and adjustably attached to said end, distal to the free end by way of an adjustable fastening mechanism. More particularly, the tube retaining member 115 has end portions of a surface disposed with a portion of an adjustable fastening mechanism that acts in cooperation with another complementary portion of the adjustable fastening mechanism disposed on a surface of the longitudinally extending strap of flexible material 114. The tube retaining member 115 serving to retain a portion of the elastomeric tubing connecting a urine bag 300 housed within the pocket 101 of the carrier 200 to a catheter inserted into a bladder of a patient fitted with the carrier 200. The tube retaining member 115 hence advantageously serves, in conjunction with the waist encircling belt 120 and strapping 130, 114 of the pocket 101, to further isolate movement of a patient from the urine bag 300 housed within the carrier 200.

According to an embodiment of the present invention, the connecting means 114 further comprises a tube holder 115 in the centre for holding and passaging of the tube of the urine bag 300. The main purpose of tube holder 115 is to stably hold the catheter tube in place while managing the length of the tube. The length of the tube of the Catheter bag can be too long hence the patient can choose to put it in the tube holders to manage the tube length. This also helps with reducing the movement of the tube which results in reduced pain.

FIG. 5a to FIG. 5f show the management of the tube length while keeping the tube intact wounded around the carrier with a help of a plurality of tube holders 108, according to the embodiments of the present invention. There can be a plurality of tube length settings that the patient can choose from to best fit as per his or her requirements. With respect to FIG. 5a, a short residual tube is shown wherein the tube is passed through three tube holders for keeping intact a longer tube. In FIG. 5b, the tube is passed through two tube holders, if the tube is little shorter. In FIG. 5c, the tube is passed through only one left side corner tube holder. In FIG. 5d, the tube is passed through the two tube holders upper and lower, present on the right side of the carrier. In FIG. 5e, the tube is passed through only one holder present on the right upper side of the carrier. FIG. 5f shows the tube not in any tube holders hence the longest possible tube length setting. This setting is recommended for times like taking a shower where the bag needs to be very far away from the body.

According to an embodiment of the present invention, the pair of horizontally present parallel supporting means 103 are made up of a flexible material to help in holding the urine bag firmly. The urine bag 300 is fastened firmly inside the pocket 101 with the help of fastening means.

According to an embodiment of the present invention, the fastening means comprises a velcro arrangement, snap-push buttons, or hook fasteners. The fastening mechanism is a riveting snap fastening mechanism comprising a male member forming one part and a female member forming another part.

According to an embodiment of the present invention, the free ends portions 120a, 120b of the waist encircling belt 120, are disposed with a plurality of hook and loop fasteners 122a, 122b, which exemplarily encompass Velcro®. More particularly, in accordance to an embodiment, one free end portion 120a of the waist encircling belt 120 is disposed with a plurality of hook fasteners 122b and the other free end portion 120b is disposed with a plurality of loop fasteners 122a. Aforementioned waist encircling belt 120 in accordance to an embodiment of the present invention comprises flexible woven material such as woven orthopaedic cotton canvas fabric. Such an arrangement, of a plurality of hook and loop fasteners 122a, 122b as elucidated above, advantageously allows the waist encircling belt 120 of the carrier 200 to be easily remove-able and to be adjustable to an individual patient's or person's waist measurement and comfort level.

According to an embodiment of the present invention, the waist encircling belt 120 for encircling a waist of a person or patient, an alternative to the adjustable fastener mechanism comprising the plurality of hook and loop fasteners 122a, 122b disposed on the free ends 120a, 120b which can be easily and conveniently fastened and unfastened and furthermore allows adjustable encircling of the waist of a patient to ensure the waist encircling belt 120 snugly fits to the patient's waist, may be employed. Exemplarily, this adjustable fastening mechanism may take the form of a slip buckle 123 (see embodiment of the carrier 200 for a urine bag 200 of the present invention alluded to in FIGS. 3a-3b and 4). More particularly, in accordance to an exemplary embodiment of the present invention, a first free end 120a of the waist encircling belt 120 is affixed to a hook portion (not explicitly shown in the appended set of figures) of said slip buckle 123 and the other free end 120b of the waist encircling belt 120 is fed through a loop portion (not explicitly shown in the appended set of figures) of said slip buckle 123, with the length of material of the waist encircling belt 120 encircling the waist of a patient or person being adjustable by controlling the length of material of the waist encircling belt 120 that is threaded through the loop portion. Exemplarily, in accordance to an alternative embodiment of the waist encircling belt 120 of the present invention, said waist encircling belt 120 is formed of a 1.5-inch-wide all cotton orthopaedic belting since it is strong enough to support a weight of a filled urine bag 300.

According to an embodiment of the present invention, the pocket 101 includes a pair flexible elastic straps 130 extending laterally from a first longitudinal side edge 106a and a second symmetrically opposing longitudinal side edge 106b. In accordance to an embodiment of the present invention, the free ends 130a of said pair of laterally extending flexible elastic straps 130 that extend laterally from the first longitudinal side edge 106a of the pocket 101 of the carrier 200 for a urine bag 300 is disposed with a plurality of hook fasteners 132b and the pair of laterally extending flexible elastic straps 130 that extend laterally from the second longitudinal side edge 106b are disposed with a plurality of loop fasteners 132a. Aforementioned laterally extending flexible elastic straps 130 having free ends 130a disposed with the plurality of hook fastener 132b and aforementioned laterally extending flexible elastic straps 130 having free ends 130a disposed with the plurality of loop fasteners 132a act in cooperation to adjustably and remove-ably secure the pouch 100 of the carrier 200 for a urine bag 300 in accordance to embodiments of the present invention alluded to herein, to a thigh of a patient or person and further reinforces the securing of the pouch 100 of the carrier 200 effected by the combination of the waist belt 120 and the strap of flexible elastic material 114 longitudinally extending from the rear panel 104 of the pouch 100 on said patient or person's thigh. More particularly, the adjustable fastening mechanism comprising of the plurality of hook fasteners 132b disposed on the free ends 130a of the pair of laterally extending flexible elastic straps 130 that extend from the first longitudinal side edge 106a and the plurality of loop fasteners 132a disposed on the free ends 130a of the pair of laterally extending flexible elastic straps 130 that extend from the second longitudinal side edge 106b of the pouch 100 are configured to adjustably and circumferentially secure said pairs of laterally extending flexible elastic straps 130 to a patient's or person's thigh and hence transmit part of the weight of the pouch 100 to the patient's or person's thigh. Moreover, aforementioned pairs of flexible elastic straps 130 were chosen to be elastic to allow stretching of said pairs of flexible elastic straps 130 to hence accommodate warping of the pouch 100 of the carrier 200 that results when a urine bag 300 housed within the pouch 100 becomes filled to its full fluid retaining capacity or is filled close to its full fluid retaining capacity. This advantageously prevents interference and restriction of blood circulation, as would be the case when said pairs of laterally extending straps 130 are not elastic.

As will be readily apparent to those skilled in the art, the present invention may easily be produced in other specific forms without departing from its essential characteristics. The present embodiments is, therefore, to be considered as merely illustrative and not restrictive, the scope of the invention being indicated by the claims rather than the foregoing description, and all changes which come within therefore intended to be embraced therein.

What is claimed is:

1. A carrier 200 for urine bag comprises:
a carrier body 100;
at least two extended arms 130, wherein the at least two extended arms 130 are present on the carrier body 100;
a pocket 101, wherein the pocket 101 is present in the carrier body 100 for placing a urine bag 300, wherein the pocket 101 is formed by a pair of horizontally present parallel supporting means 103 and a plurality of vertical supporters 106 on a front side, and a rear panel 104 on a rear side;
an opening 201 for passaging of a drainage valve;
a belt 120; and
a connecting means 114, wherein the connecting means connects the carrier body 100 with the belt 120.

2. The carrier for urine bag as claimed in claim 1, wherein a plurality of tube holders 108 are present over the corners of the carrier body 100.

3. The carrier for urine bag as claimed in claim 1, wherein the at least two extended arms 130 are a top extended arm and a bottom extended arm.

4. The carrier for urine bag as claimed in claim 1, wherein the at least two extended arms 130 are wrapped around a patient's body and are fixed with the help of a fastening means present on the horizontal supporting means 103 of the carrier body 100.

5. The carrier for urine bag as claimed in claim 1, wherein the pocket 101 is covered with a netting fabric internally.

6. The carrier for urine bag as claimed in claim 1, wherein the connecting means 114 comprises a distance adjusting mechanism 116 for adjusting the distance between the belt 120 and the carrier body 100.

7. The carrier for urine bag as claimed in claim 1, wherein the connecting means 114 further comprises a tube holder 115 in the centre for holding and passaging of the tube of the urine bag 300.

8. The carrier for urine bag as claimed in claim 1, wherein the pair of horizontally present parallel supporting means 103 are made up of a flexible material to help in holding the urine bag firmly.

9. The carrier for urine bag as claimed in claim 1, wherein the urine bag 300 is fastened firmly inside the pocket 101 with the help of fastening means.

10. The carrier for urine bag as claimed in claim 1, wherein the fastening means comprises a velcro arrangement, snap-push buttons, or hook fasteners.
